(19) United States
(12) Patent Application Publication
(10) Pub. No.: US 2022/0160867 A1
(43) Pub. Date: May 26, 2022

(54) METHODS AND COMPOSITIONS COMPRISING CATIONIC LIPIDS FOR IMMUNOTHERAPY BY DIRECT TUMOR INJECTION

(71) Applicant: PDS Biotechnology Corporation, Berkeley Heights, NJ (US)

(72) Inventors: Frank Bedu-Addo, Stamford, CT (US); Greg Conn, Madrid (ES); Martin Ward, Lexington, KY (US); Jerold Woodward, Lexington, KY (US); Siva K. Gandhapudi, Blue Ash, OH (US)

(21) Appl. No.: 17/532,613

(22) Filed: Nov. 22, 2021

Related U.S. Application Data
(60) Provisional application No. 63/116,406, filed on Nov. 20, 2020.

(51) Int. Cl. A61K 39/39; A61K 39/00

(52) U.S. Cl. CPC A61K 39/39; A61K 2039/6018; A61K 39/0011

(57) ABSTRACT

Provided herein are novel immunotherapeutic interventions comprising the use of cationic lipid-based compositions for direct tumor injection. The compositions are effective for reducing, eliminating and/or preventing tumor growth and cancer proliferation with local, targeted, systemic and distal effectiveness. The compositions may comprise one or more cationic lipids such as DOTAP and DOTMA, and may further comprise additional components such as antigens, therapeutic agents and/or pharmaceutically acceptable excipients.

### TECHNICAL FIELD

Embodiments of the present disclosure relate generally to novel immunotherapeutic interventions, in particular, the use of cationic lipid-based vaccines, compositions and methods of use thereof, for direct tumor injection.

### BACKGROUND OF THE INVENTION

A number of studies evaluating direct tumor injection as a means to generate anti-tumor immune responses at local and distal tumor sites have been evaluated in the clinic. Such agents include Bacillus Calmette-Guerin (BCG), oncolytic viruses, IL-2, small molecule STING agonist, toll receptor agonists and local irradiation of tumors. Direct intra-tumoral injection of oncolytic viruses has recently been approved for the treatment of metastatic melanoma. Intra-tumoral injection generally defined as direct injection of immunostimulatory agents into the tumor itself, has the potential to result in superior priming of an antitumor response. Furthermore, direct injection into the tumor could not only reduce systemic exposure, off-target toxicities, and the amounts of drug used but also induce stronger antitumor activity in the injected tumor lesion and maybe in distant noninjected tumor lesions as well.¹ Topical toll-like-receptor (TLR) agonists have been studied for their use in the treatment of cancer. Imiquimod, which is a TLR-7/8 agonist, has demonstrated clinical antitumor activity and is approved for the treatment of superficial basal cell carcinomas, actinic keratosis, and genital warts.² In a reported phase I/II trial topical imiquimod in combination with intra-lesional interleukin (IL)-2, 13 patients with cutaneous melanoma metastases were tested. A total of 182 tumor lesions were treated and anti-tumor responses reported in 92/182 lesions, with complete regression of 74 lesions. In a separate study, Kidner et al.³ reported that in a clinical trial combining intralesional BCG with topical imiquimod in 9 melanoma patients, 5/9 patients experienced complete clinical benefit. Another topical TLR-7/8 agonist, Resiquimod, has been studied in a phase I trial of 12 patients with stage IA-IIA cutaneous T-cell lymphoma (CTCL) by Rook et al.⁴ Partial benefit was reported in 75% of patients and full clinical benefit seen in 30% of the patients. In this study, T-cell receptor sequencing and flow cytometry demonstrated a decrease in clonal malignant T cells in 90% of the patients and a complete eradication in 30%.

In other studies, intratumoral TLR agonists have been tested in combination with mild (2×2 Gy) local irradiation in patients with B- and T-cell lymphomas.⁵ Brody et al. reported an objective response rate of in 4/15 patients in noninjected target lesions. An additional eight patients showed durable stable disease. Kim et al.⁶ reported an objective response rate in 5/14 mycosis fungoides patients when the same combination therapy in noninjected (abscopal) target lesions. In the biopsies carried out at injected sites, they found a significant decrease in CD25+/FoxP3+ T cells and antigen-presenting cells, and increased CD123+ pDCs upon intratumoral immunization.

It has also been reported that local tissue damage and inflammation induced by radiotherapy can generate tumor antigens and release danger-associated molecular patterns.⁷ Like intra-tumoral drugs, local irradiation may induce systemic immune changes such as an increase in the levels of systemic cytokines and chemokines.⁸ It has also been reported that irradiation efficacy partly relies on the immune system and may generate antitumor immunity through immunogenic cell death, antigen release, MHC-I upregulation, and T-cell responses.⁹ It is also however suggested that radiotherapy may not address existing immune tolerance against tumor antigens. It is also proposed that after initial tumor tissue damage, negative feedback loops such as Treg proliferation will effectively restore immune to cytotoxic T cells.¹⁰

Intra-tumoral injection of cytokines is also being studied as a cancer immunotherapy approach. IL-2 cytokine therapy is currently used to treat melanoma.¹¹ The clinical activity of intralesional IL-2 is most beneficial in the smaller stage III melanomas.¹² A combination of intralesional IL-2 with anti-CTLA-4 has been reported in a small phase I trial has been reported. Responses were seen in 67% of patients and an objective response rate by irRC in 40%.¹³

Though significant strides have been made in the rational design of vaccines and cancer immunotherapy there continues to be an ongoing need for the development of cancer treatments both prophylactic and therapeutic. There is a need for the development of compositions that are both specific and effective with minimal side-effects.

### SUMMARY OF THE INVENTION

Disclosed herein are novel methods for inducing an anti-tumor immune response by direct intra-tumoral injection of a composition comprising one or more cationic lipids. In certain embodiments, the one or more cationic lipids comprises at least one non-steroidal lipid. In certain embodiments, the one or more cationic lipids comprises 1,2-dioleoyl-3-trimethylammonium propane (DOTAP), N-1-(2,3-dioleoyloxy)-propyl-N,N,N-trimethyl ammonium chloride (DOTMA), 1,2-dioleoyl-sn-glycero-3-ethylphosphocholine (DOEPC), and combinations thereof.

### BRIEF DESCRIPTION OF FIGURES

FIG. 1 provides a survival plot: B6 mice (n=4 per group) were implanted with 50,000 TC1 tumor cells subcutaneously. On day 10, group 2 received tumor vaccine R-DOTAP−HPV mix formulation (100 μl) (ASP3-250-HPV mix) containing HPV antigens (ASP3/R-DOTAP (S.C.) in the opposite flank of the tumor and group 3 mice received intra-tumoral injection of R-DOTAP (50 μl of 6 mg/ml) (RDOTAP (IT)).

### DETAILED DESCRIPTION OF THE INVENTION

The following detailed description is exemplary and explanatory and is intended to provide further explanation of the present disclosure described herein. Other advantages, and novel features will be readily apparent to those skilled in the art from the following detailed description of the present disclosure.

The texts of references mentioned herein along with the following patents and patent applications are incorporated herein in their entirety: U.S. Pat. No. 7,303,881 issued Dec. 4, 2007, U.S. Pat. No. 8,877,206 issued Nov. 4, 2014, U.S. Pat. No. 9,789,129 issued Oct. 17, 2017, U.S. patent application Ser. No. 14/344,327 filed Nov. 5, 2014, U.S. patent application Ser. No. 14/407,419 filed Dec. 11, 2014, U.S. patent application Ser. No. 14/429,123 filed Mar. 18, 2015, U.S. patent application Ser. No. 15/725,985 filed Oct. 5, 2017, U.S. patent application Ser. No. 15/724,818 filed Oct. 4, 2017, U.S. Provisional Patent Application No. 62/633,865 filed Feb. 22, 2018, U.S. Provisional Patent Application No. 62/809,182 filed Feb. 22, 2019, U.S. Provisional Patent Application No. 62/939,161 filed Nov. 22, 2019, and U.S. Provisional Patent Application No. 63/116,406 filed Nov. 20, 2020.

There is growing interest in the direct injection of tumors with agents capable of stimulating cellular immune responses against the tumors. The goal of most such approaches is to utilize the presence of tumor antigens already present within the tumors to generate antitumor immunity against cancer cell antigens. This approach essentially uses the tumor as its own vaccine. Direct tumor injection may also aid in the generation of a polyclonal antitumor immune response against multiple cancer targets. This is important in increasing the potential to better address the heterogeneity of cancer. A significant focus of direct tumor injection is the potential to be agnostic from the nature of the most highly immunogenic tumor antigens [neo-antigens, glycopeptides, tumor-associated carcino-embryonic antigens, major histocompatibility complex (MHC) I or II restricted].

The inherent heterogenous nature of any cancer is a result of the development and accumulation of mutations in the cancer cell genome over time. It is well established that any cancer cell can develop mutations that are absent in the parent cancer cell. Such new mutations may accumulate with time and the resulting mutation profile may differ across tumor lesions. Intratumoral immunotherapy presents strong potential to generate antitumor immune responses against the full repertoire of tumor cell subclones present in a tumor. The ability provided by direct tumor injection to directly inject in a single patient multiple tumor lesions should significantly enhance the possibility of generating a polyclonal immune response targeting a broad range of antigens shared by all the cancer cells. It has also been discovered that the possibility of generating both B-cell and T-cell antitumor immune responses upon intratumoral immunotherapy may overcome some of the escape mechanisms seen with ICT mAbs monotherapies [e.g. loss of human leukocyte antigen (HLA)-I expression on cancer cells].

Direct tumor injection presents significant advantages over traditional cancer vaccines. Dendritic cell vaccines for example have to be pulsed with pre-identified tumor antigens which must be isolated and produced. Recently, neo-antigen vaccines have received significant attention. Such vaccines also require multiple development steps including tumor biopsy, tumor sequencing, epitope binding prediction and GMP production of the epitopes. For traditional cancer vaccines, there is usually some uncertainty regarding the most immunogenic targets for the specific cancer/patient. Such vaccines are also limited in the number of antigens that can be successfully presented, and therefore in their ability to generate polyclonal immunity. Several cancer vaccines are based on a HLA-restricted single epitope CD8+ peptides which limits the ability to generate broadly applicable immune responses.

The inventors herein provide novel compositions and methods comprising cationic lipids as for generating broad a robust anti-tumor immune responses against multiple tumor antigens by direct injection of the lipids into a tumor.

Disclosed herein are novel anti-cancer methods comprising the use of intra-tumoral immunotherapy as an immunotherapeutic strategy wherein a tumor is utilized as a contributor to its own vaccine. Local and site-specific delivery of immunotherapeutic drugs allow for the use of multiple combination therapies, while preventing significant systemic exposure and commonly observed off-target toxicities and side-effects. Upon direct injection into the tumor, a high concentration of immunostimulatory products may be delivered in situ. Furthermore, as is often typical for many cancers, even when there is a lack of knowledge regarding the dominant epitopes of a given cancer, a direct tumor injection may be utilized to induce an immune response against the relevant neo-antigens or tumor-associated antigens without a requirement for their prior identification or characterization. As detailed in the Example section herein, cationic lipids were studied for their ability to induce both local and distal anti-tumor immune responses upon direct tumor injection without the use of an antigen. The resulting cationic lipid-induced immune activation within the tumors induced a strong priming of cancer immunity locally, while also generating distal anti-tumor responses.

As previously discovered by the inventors, cationic lipids such as R-DOTAP can efficiently prime antigen-presenting T cells by delivering antigen cargo into the antigen-presenting cells and inducing type I interferons necessary for optimal T cell activation. At certain concentrations, cationic lipids show cytotoxic effects and membrane destabilization. As provided herein, the inventors have now discovered that direct intra-tumoral injection of optimal doses of cationic lipids will cause tumor cell death as well as the release of tumor antigens that will interact with cationic lipids and be taken up by the antigen-presenting cells. The cationic lipids as administered according to the invention, also induce type I interferons by the antigen-loaded dendritic cells in the local tumor micro-environment and the draining lymph node, and trigger T cell priming. Therefore, when delivered as monotherapy or in combination with other systemic or intra-tumoral immunotherapies, cationic lipids can generate an antitumor immune response to regress tumors locally and at distinct sites.

Provided herein are novel methods for inducing an anti-tumor immune response by direct intra-tumoral injection of a composition comprising one or more cationic lipids. In an embodiment, the cationic lipids comprise at least one non-steroidal lipid. The cationic lipids may comprise 1,2-dioleoyl-3-trimethylammonium propane (DOTAP), N-1-(2,3-dioleoyloxy)-propyl-N,N,N-trimethyl ammonium chloride (DOTMA), 1,2-dioleoyl-sn-glycero-3-ethylphosphocholine (DOEPC), and combinations thereof. In certain embodiments, the cationic lipids comprise an enantiomer of a cationic lipid selected from the group consisting of, but not limited to, R-DOTAP, R-DDA, R-DOEPC, R-DOTMA, S-DOTAP, S-DDA, S-DOEPC, S-DOTMA, variations or analogs thereof. In certain embodiments, the enantiomer is (R)-1,2-dioleoyl-3-trimethylammonium propane (R-DOTAP).

In certain embodiments, the composition administered via intra-tumoral injection comprises one or more cationic lipids and further comprises one or more antigens. The one or more antigens may comprise a protein, peptide, polysaccharide, glycoprotein, glycolipid, nucleic acid, or combination thereof. The antigen may comprise a viral antigen, a bacterial antigen, a pathogenic antigen, microbial antigen, cancer antigen and active fragments, isolates and combinations thereof. The antigen may comprise a lipoprotein, a lipopeptide, or a protein or peptide modified with an amino acid sequence having an increased hydrophobicity or a decreased hydrophobicity.

In certain embodiments, the composition administered via intra-tumoral injection comprises one or more cationic lipids, may optionally comprise one or more antigens and may also comprise therapeutic agents and/or pharmaceutically acceptable excipients. In certain embodiments, the compositions may be in the form of a controlled release preparation; the controlled release preparation may comprise the use of polymer complexes such as polyesters, polyamino acids, methylcellulose, polyvinyl, poly(lactic acid), and hydrogels. Administration of the compositions described herein may result in the elevation of antigen-specific CD8+ T cell responses as well as alteration of the tumor microenvironment.

Provided herein are methods for inducing an immunogenic response in a subject comprising the intra-tumoral administration of a composition comprising a cationic lipid wherein the administration of the cationic lipid results in the stimulation of an anti-tumor response. The cationic lipid may comprise 1,2-dioleoyl-3-trimethylammonium propane (DOTAP), (R)-1,2-dioleoyl-3-trimethylammonium propane (R-DOTAP) N-1-(2,3-dioleoyloxy)-propyl-N,N,N-trimethyl ammonium chloride (DOTMA), 1,2-dioleoyl-sn-glycero-3-ethylphosphocholine (DOEPC), and combinations thereof. The compositions may optionally comprise one or more antigens, and may be in the form of controlled release preparations.

### Lipid Adjuvants

Cationic lipids have been reported to have strong immune-stimulatory adjuvant effect. The cationic lipids of the present invention may form liposomes that are optionally mixed with antigen and may contain the cationic lipids alone or in combination with neutral lipids and/or other pharmaceutical excipients. Suitable cationic lipid species include: 3-β[⁴N—(¹N,⁸-diguanidino spermidine)-carbamoyl] cholesterol (BGSC); 3-β[N,N-diguanidinoethyl-aminoethane)-carbamoyl] cholesterol (BGTC); N,N¹N²N³Tetra-methyltetrapalmitylspermine (cellfectin); N-t-butyl-N′-tetradecyl-3-tetradecyl-aminopropion-amidine (CLONfectin); dimethyldioctadecyl ammonium bromide (DDAB); 1,2-dimyristyloxypropyl-3-dimethyl-hydroxy ethyl ammonium bromide (DMRIE); 2,3-dioleoyloxy-N-[2(sperminecarboxamido)ethyl]-N,N-dimethyl-1-p-ropanaminium trifluorocetate) (DOSPA); 1,3-dioleoyloxy-2-(6-carboxyspermyl)-propyl amide (DOSPER); 4-(2,3-bis-palmitoyloxy-propyl)-1-methyl-1H-imidazole (DPIM) N,N,N′,N′-tetramethyl-N,N′-bis(2-hydroxyethyl)-2,3 dioleoyloxy-1,4-butanediammonium iodide) (Tfx-50); N-1-(2,3-dioleoyloxy) propyl-N,N,N-trimethyl ammonium chloride (DOTMA) or other N—(N,N-1-dialkoxy)-alkyl-N,N,N-trisubstituted ammonium surfactants; 1,2 dioleoyl-3-(4′-trimethylammonio) butanol-sn-glycerol (DOBT) or cholesteryl (4′trimethylammonia) butanoate (ChOTB) where the trimethylammonium group is connected via a butanol spacer arm to either the double chain (for DOTB) or cholesteryl group (for ChOTB); DORI (DL-1,2-dioleoyl-3-dimethylaminopropyl-β-hydroxyethylammonium) or DORIE (DL-1,2-O-dioleoyl-3-dimethylaminopropyl-β-hydroxyethylammonium) (DORIE) or analogs thereof as disclosed in WO 93/03709; 1,2-dioleoyl-3-succinyl-sn-glycerol choline ester (DOSC); cholesteryl hemisuccinate ester (ChOSC); lipopolyamines such as dioctadecylamidoglycylspermine (DOGS) and dipalmitoyl phosphatidylethanolamylspermine (DPPES) or the cationic lipids disclosed in U.S. Pat. No. 5,283,185, cholesteryl-3β-carboxyl-amido-ethylenetrimethylammonium iodide, 1-dimethylamino-3-trimethylammonio-DL-2-propyl-cholesteryl carboxylate iodide, cholesteryl-3-O-carboxyamidoethyleneamine, cholesteryl-3-β-oxysuccinamido-ethylenetrimethylammonium iodide, 1-dimethylamino-3-trimethylammonio-DL-2-propyl-cholesteryl-3-β-oxysuccinate iodide, 2-(2-trimethylammonio)-ethylmethylamino ethyl-cholesteryl-3-β-oxysuccinate iodide, 3-β-N—(N′,N′-dimethylaminoethane) carbamoyl cholesterol (DC-chol), and 3-β-N-(polyethyleneimine)-carbamoylcholesterol; O,O′-dimyristyl-N-lysyl aspartate (DMKE); O,O′-dimyristyl-N-lysyl-glutamate (DMKD); 1,2-dimyristyloxypropyl-3-dimethyl-hydroxy ethyl ammonium bromide (DMRIE); 1,2-dilauroyl-sn-glycero-3-ethylphosphocholine (DLEPC); 1,2-dimyristoyl-sn-glycero-3-ethylphosphocholine (DMEPC); 1,2-dioleoyl-sn-glycero-3-ethylphosphocholine (DOEPC); 1,2-dipalmitoyl-sn-glycero-3-ethylphosphocholine (DPEPC); 1,2-di stearoyl-sn-glycero-3-ethylphosphocholine (DSEPC); 1,2-dioleoyl-3-trimethylammoninum propane (DOTAP); dioleoyl dimethylaminopropane (DODAP); 1,2-palmitoyl-3-trimethylammonium propane (DPTAP); 1,2-di stearoyl-3-trimethylammonium propane (DSTAP), 1,2-myristoyl-3-trimethylammonium propane (DMTAP); and sodium dodecyl sulfate (SDS). The present invention contemplates the use of structural variants and derivatives of the cationic lipids disclosed in this application.

Certain aspects of the present invention include non-steroidal chiral cationic lipids having a structure represented by the following formula:

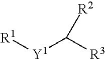

wherein in R¹ is a quaternary ammonium group, Y¹ is chosen from a hydrocarbon chain, an ester, a ketone, and a peptide, R² and R³ are independently chosen from a saturated fatty acid, an unsaturated fatty acid, an ester-linked hydrocarbon, phosphor-diesters, and combinations thereof. DOTAP, DMTAP, DSTAP, DPTAP, DPEPC, DSEPC, DMEPC, DLEPC, DOEPC, DMKE, DMKD, DOSPA, DOTMA, are examples of lipids having this general structure.

In one embodiment, chiral cationic lipids of the invention are lipids in which bonds between the lipophilic group and the amino group are stable in aqueous solution. Thus, an attribute of the complexes of the invention is their stability during storage (i.e., their ability to maintain a small diameter and retain biological activity over time following their formation). Such bonds used in the cationic lipids include amide bonds, ester bonds, ether bonds and carbamoyl bonds. Those of skill in the art would readily understand that liposomes containing more than one cationic lipid species may be used to produce the complexes of the present invention. For example, liposomes comprising two cationic lipid species, lysyl-phosphatidylethanolamine and β-alanyl cholesterol ester have been disclosed for certain drug delivery applications [Brunette, E. et al., Nucl. Acids Res., 20:1151 (1992)].

It is to be further understood that in considering chiral cationic liposomes suitable for use in the invention and optionally mixing with one more antigens, the methods of the invention are not restricted only to the use of the cationic lipids recited above but rather, any lipid composition may be used so long as a cationic liposome is produced and the resulting cationic charge density is sufficient to activate and induce an immune response.

Thus, the lipids of the invention may contain other lipids in addition to the cationic lipids. These lipids include, but are not limited to, lyso lipids of which lysophosphatidylcholine (1-oleoyl lysophosphatidylcholine) is an example, cholesterol, or neutral phospholipids including dioleoyl phosphatidyl ethanolamine (DOPE) or dioleoyl phosphatidylcholine (DOPC) as well as various lipophylic surfactants, containing polyethylene glycol moieties, of which Tween-80 and PEG-PE are examples.

The cationic lipids of the invention may also contain negatively charged lipids as well as cationic lipids so long as the net charge of the complexes formed is positive and/or the surface of the complex is positively charged. Negatively charged lipids of the invention are those comprising at least one lipid species having a net negative charge at or near physiological pH or combinations of these. Suitable negatively charged lipid species include, but are not limited to, CHEMS (cholesteryl hemisuccinate), NGPE (N-glutaryl phosphatidlylethanolanine), phosphatidyl glycerol and phosphatidic acid or a similar phospholipid analog.

Methods for producing the liposomes to be used in the production of the lipid comprising drug delivery complexes of the present invention are known to those of ordinary skill in the art. A review of methodologies of liposome preparation may be found in Liposome Technology (CFC Press New York 1984); Liposomes by Ostro (Marcel Dekker, 1987); Methods Biochem Anal. 33:337-462 (1988) and U.S. Pat. No. 5,283,185. Such methods include freeze-thaw extrusion and sonication. Both unilamellar liposomes (less than about 200 nm in average diameter) and multilamellar liposomes (greater than about 300 nm in average diameter) may be used as starting components to produce the complexes of this invention.

In the cationic liposomes utilized to produce the cationic lipid vaccines of this invention, the cationic lipid is present in the liposome at from about 10 mole % to about 100 mole % of total liposomal lipid, or from about 20 mole % to about 80 mole %. The neutral lipid, when included in the liposome, may be present at a concentration of from about 0 mole % to about 90 mole % of the total liposomal lipid, or from about 20 mole % to about 80 mole %, or from 40 mole % to 80 mole %. The negatively charged lipid, when included in the liposome, may be present at a concentration ranging from about 0 mole % to about 49 mole % of the total liposomal lipid, or from about 0 mole % to about 40 mole %. In one embodiment, the liposomes contain a cationic and a neutral lipid, in ratios between about 2:8 to about 6:4. It is further understood that the complexes of the present invention may contain modified lipids, protein, polycations or receptor ligands which function as a targeting factor directing the complex to a particular tissue or cell type. Examples of targeting factors include, but are not limited to, asialoglycoprotein, insulin, low density lipoprotein (LDL), folate and monoclonal and polyclonal antibodies directed against cell surface molecules. Furthermore, to modify the circulatory half-life of the complexes, the positive surface charge can be sterically shielded by incorporating lipophilic surfactants which contain polyethylene glycol moieties.

The cationic lipid compositions of the invention may be stored in isotonic sucrose or dextrose solution upon collection from the sucrose gradient or they may be lyophilized and then reconstituted in an isotonic solution prior to use. In one embodiment, the cationic lipid complexes are stored in solution. The stability of the cationic lipid complexes of the present invention is measured by specific assays to determine the physical stability and biological activity of the cationic lipid vaccines over time in storage. The physical stability of the cationic lipid compositions is measured by determining the diameter and charge of the cationic lipid complexes by methods known to those of ordinary skill in the art, including for example, electron microscopy, gel filtration chromatography or by means of quasi-elastic light scattering using, for example, a Coulter N4SD particle size analyzer. The physical stability of the cationic lipid complex is “substantially unchanged” over storage when the diameter of the stored cationic lipid vaccines is not increased by more than 100%, or by not more than 50%, or by not more than 30%, over the diameter of the cationic lipid complexes as determined at the time the cationic lipid vaccines were purified.

While it is possible for the cationic lipid to be administered in a pure or substantially pure form, it certain embodiments it may be administered as a pharmaceutical composition, formulation or preparation. Pharmaceutical formulations using the chiral cationic lipid complexes of the invention may comprise the cationic lipid vaccines in a physiologically compatible sterile buffer such as, for example, phosphate buffered saline, isotonic saline or low ionic strength buffer such as acetate or Hepes (an exemplary pH being in the range of about 5.0 to about 8.0). The chiral cationic lipid compositions may be administered as liquid solutions for intratumoral, intraarterial, intravenous, intratracheal, intraperitoneal, subcutaneous, and intramuscular administration.

In various embodiments described herein, the composition further comprises one or more antigens. As used herein, the term “antigen” refers to any agent (e.g., protein, peptide, polysaccharide, glycoprotein, glycolipid, nucleic acid, or combination thereof) that, when introduced into a mammal having an immune system (directly or upon expression as in, e.g., DNA vaccines), is recognized by the immune system of the mammal and is capable of eliciting an immune response. As defined herein, the antigen-induced immune response can be humoral or cell-mediated, or both. An agent is termed “antigenic” when it is capable of specifically interacting with an antigen recognition molecule of the immune system, such as an immunoglobulin (antibody) or T cell antigen receptor (TCR).

In some embodiments, one or more antigens is a protein-based antigen. In other embodiments, one or more antigens is a peptide-based antigen. In various embodiments, one or more antigens is selected from the group consisting of a viral antigen, a bacterial antigen, and a pathogenic antigen. A “microbial antigen,” as used herein, is an antigen of a microorganism and includes, but is not limited to, infectious virus, infectious bacteria, infectious parasites and infectious fungi. Microbial antigens may be intact microorganisms, and natural isolates, fragments, or derivatives thereof, synthetic compounds which are identical to or similar to naturally-occurring microbial antigens and, preferably, induce an immune response specific for the corresponding microorganism (from which the naturally-occurring microbial antigen originated). In one embodiment, the antigen is a cancer antigen. In one embodiment, the antigen is a viral antigen. In another embodiment, the antigen is a fungal antigen. In another embodiment, the antigen is a bacterial antigen. In various embodiments, the antigen is a pathogenic antigen. In some embodiments, the pathogenic antigen is a synthetic or recombinant antigen.

In some embodiments, the pathogenic antigen is a synthetic or recombinant antigen. In some embodiments, the antigen is a cancer antigen. A “cancer antigen,” as used herein, is a molecule or compound (e.g., a protein, peptide, polypeptide, lipoprotein, lipopeptide, glycoprotein, glycopeptides, lipid, glycolipid, carbohydrate, RNA, and/or DNA) associated with a tumor or cancer cell and which is capable of provoking an immune response (humoral and/or cellular) when expressed on the surface of an antigen presenting cell in the context of an MHC molecule. For example, a cancer antigen may be a tumor-associated antigen. Tumor-associated antigens include self-antigens, as well as other antigens that may not be specifically associated with a cancer, but nonetheless enhance an immune response to and/or reduce the growth of a tumor or cancer cell when administered to a mammal. In one embodiment.

In various embodiments, at least one antigen is selected from the group consisting of a lipoprotein, a lipopeptide, and a protein or peptide modified with an amino acid sequence having an increased hydrophobicity or a decreased hydrophobicity. In some embodiments, one or more antigens is an antigen modified to increase hydrophobicity of the antigen. In one embodiment, at least one antigen is a modified protein or peptide. In some embodiments, the modified protein or peptide is bonded to a hydrophobic group. In other embodiments, the modified protein or peptide bonded to a hydrophobic group further comprises a linker sequence between the antigen and the hydrophobic group. In some embodiments, the hydrophobic group is a palmitoyl group. In yet other embodiments, at least one antigen is an unmodified protein or peptide.

### Formulations

The formulations of the present invention may incorporate any stabilizer known in the art. Illustrative stabilizers are cholesterol and other sterols that may help rigidify the liposome bilayer and prevent disintegration or destabilization of the bilayer. Also agents such as polyethylene glycol, poly-, and mono-saccharides may be incorporated into the liposome to modify the liposome surface and prevent it from being destabilized due to interaction with blood-components. Other illustrative stabilizers are proteins, saccharides, inorganic acids, or organic acids which may be used either on their own or as admixtures.

A number of pharmaceutical methods may be employed to control, modify, or prolong the duration of immune stimulation. Controlled release preparations may be achieved through the use of polymer complexes such as polyesters, polyamino acids, methylcellulose, polyvinyl, poly(lactic acid), and hydrogels to encapsulate or entrap the cationic lipids and slowly release them. Similar polymers may also be used to adsorb the liposomes. The liposomes may be contained in emulsion formulations in order to alter the release profile of the stimulant. Alternatively, the duration of the stimulant's presence in the blood circulation may be enhanced by coating the surface of the liposome with compounds such as polyethylene glycol or other polymers and other substances such as saccharides which are capable of enhancing the circulation time or half-life of liposomes and emulsions.

When oral preparations are required, the chiral cationic lipids may be combined with typical pharmaceutical carriers known in the art such as, for example, sucrose, lactose, methylcellulose, carboxymethyl cellulose, or gum Arabic, among others. The cationic lipids may also be encapsulated in capsules or tablets for systemic delivery.

Administration of the chiral cationic lipid compositions of the present disclosure may be for either a prophylactic or therapeutic purpose. When provided prophylactically, the cationic lipid is provided in advance of any evidence or symptoms of illness. When provided therapeutically, the cationic lipid is provided at or after the onset of disease or manifestation of a tumor. The therapeutic administration of the immune-stimulant serves to attenuate or cure the disease. For both purposes, the cationic lipid may be administered with an additional therapeutic agent(s) or antigen(s). When the cationic lipids are administered with an additional therapeutic agent or antigen, the prophylactic or therapeutic effect may be generated against a specific disease, including for example, disease or disorders caused by microbes.

The formulations of the present invention, both for veterinary and for human use, comprise a pure chiral cationic lipid alone as described above, as a mixture of R and S enantiomers, with one or more therapeutic ingredients such as an antigen(s) or drug molecule(s). The formulations may conveniently be presented in unit dosage form and may be prepared by any method known in the pharmaceutical art.

### Terms

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively.

As used herein, the term “about” means a variability of 10% from the reference given, unless otherwise specified.

As used herein, the terms “subject” and “patient” are used interchangeably and include a mammal, e.g., a human, mouse, rat, guinea pig, dog, cat, horse, cow, pig, or non-human primate, such as a monkey, chimpanzee, baboon or gorilla.

As used herein, the terms “disease”, “disorder” and “condition” are used interchangeably, to indicate an abnormal state in a subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

The compositions of the disclosure comprise an amount of a composition cationic lipids that is effective for generating an immunogenic response in a subject. Specifically, the dosage of the composition to achieve a therapeutic effect will depend on factors such as the formulation, pharmacological potency of the composition, age, weight and sex of the patient, condition being treated, severity of the patient's symptoms, route of delivery, and response pattern of the patient. It is also contemplated that the treatment and dosage of the compositions may be administered in unit dosage form and that one skilled in the art would adjust the unit dosage form accordingly to reflect the relative level of activity. The decision as to the particular dosage to be employed (and the number of times to be administered per day) is within the discretion of the ordinarily-skilled physician, and may be varied by titration of the dosage to the particular circumstances to produce the therapeutic effect. Further, one of skill in the art would be able to calculate any changes in effective amounts of the compositions due to changes in the composition components or dilutions. In one embodiment, the compositions may be diluted 2-fold. In another embodiment, the compositions may be diluted 4-fold. In a further embodiment, the compositions may be diluted 8-fold.

The effective amount of the compositions disclosed herein may, therefore, be about 1 mg to about 1000 mg per dose based on a 70 kg mammalian, for example human, subject. In another embodiment, the therapeutically effective amount is about 2 mg to about 250 mg per dose. In a further embodiment, the therapeutically effective amount is about 5 mg to about 100 mg. In yet a further embodiment, the therapeutically effective amount is about 25 mg to 50 mg, about 20 mg, about 15 mg, about 10 mg, about 5 mg, about 1 mg, about 0.1 mg, about 0.01 mg, about 0.001 mg.

The effective amounts (if administered therapeutically) may be provided on regular schedule, i.e., on a daily, weekly, monthly, or yearly basis or on an irregular schedule with varying administration days, weeks, months, etc. Alternatively, the therapeutically effective amount to be administered may vary. In one embodiment, the therapeutically effective amount for the first dose is higher than the therapeutically effective amount for one or more of the subsequent doses. In another embodiment, the therapeutically effective amount for the first dose is lower than the therapeutically effective amount for one or more of the subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every 2 weeks, about every 3 weeks, about every month, about every 2 months, about every 3 months and about every 6 months. The number and frequency of dosages corresponding to a completed course of therapy will be determined according to the judgment of a health-care practitioner.

The compositions may be administered by any route, taking into consideration the specific condition for which it has been selected. In certain embodiments, the compositions are administered via intra-tumoral injection. In alternative embodiments, The compositions may be delivered to a tumor orally (for example in the case of oral, throat, or esophageal cancer), by injection, inhalation (including orally, intranasally and intratracheally), ocularly, transdermally (via simple passive diffusion formulations or via facilitated delivery using, for example, iontophoresis, microporation with microneedles, radio-frequency ablation or the like), intravascularly, cutaneously, subcutaneously, intramuscularly, sublingually, intracranially, epidurally, rectally, intravesically, and vaginally, among others.

The compositions may be formulated neat or with one or more pharmaceutical carriers and/or excipients for administration. The amount of the pharmaceutical carrier(s) is determined by the solubility, the chemical nature of the cationic lipid being employed, chosen route of administration and standard pharmacological practice. The pharmaceutical carrier(s) may be solid or liquid and may incorporate both solid and liquid carriers/matrices. A variety of suitable liquid carriers is known and may be readily selected by one of skill in the art. Such carriers may include, e.g., dimethylsulfoxide (DMSO), saline, buffered saline, cyclodextrin, hydroxypropylcyclodextrin (HPβCD), n-dodecyl-β-D-maltoside (DDM) and mixtures thereof. Similarly, a variety of solid (rigid or flexible) carriers and excipients are known to those of skill in the art.

Although the compositions may be administered alone, they may also be administered in the presence of one or more pharmaceutical carriers that are physiologically compatible. The carriers may be in dry or liquid form and must be pharmaceutically acceptable. Liquid pharmaceutical compositions may be sterile solutions or suspensions. When liquid carriers are utilized, they may be sterile liquids. Liquid carriers may be utilized in preparing solutions, suspensions, emulsions, syrups and elixirs. In one embodiment, the compositions may be dissolved a liquid carrier. In another embodiment, the compositions may be suspended in a liquid carrier. One of skill in the art of formulations would be able to select a suitable liquid carrier, depending on the route of administration. The compositions may alternatively be formulated in a solid carrier such as a table, caplet or powder. In one embodiment, the composition may be compacted into a unit dose form, i.e., tablet or caplet. In another embodiment, the composition may be added to unit dose form, i.e., a capsule. In a further embodiment, the composition may be formulated for administration as a powder. A formulation in a solid carrier may perform a variety of functions, i.e., may perform the functions of two or more of the excipients described below or it may be delivered via injection for site-specific controlled release. A solid carrier may also act as a flavoring agent, lubricant, solubilizer, suspending agent, filler, glidant, compression aid, binder, disintegrant, or encapsulating material. In one embodiment, a solid carrier acts as a lubricant, solubilizer, suspending agent, binder, disintegrant, or encapsulating material. The composition may also be sub-divided to contain appropriate quantities of the compositions. For example, the unit dosage can be packaged compositions, e.g., packeted powders, vials, ampoules, prefilled syringes or sachets containing liquids.

In an embodiment, the compositions may be administered by a modified-release delivery device. “Modified-release” as used herein refers to delivery of the disclosed compositions which is controlled, for example over a period of at least about 8 hours (e.g., extended delivery) to at least about 12 hours (e.g., sustained delivery). Such devices may also permit immediate release (e.g., therapeutic levels achieved in under about 1 hour, or in less than about 2 hours). Those of skill in the art know suitable modified-release delivery devices.

Also provided are kits comprising the compositions disclosed herein. The kit may further comprise packaging or a container with the compositions formulated for the delivery route. Suitably, the kit contains instructions on dosing and an insert regarding the compositions.

A number of packages or kits are known in the art for dispensing pharmaceutical compositions for periodic use. In one embodiment, the package has indicators for each period. In another embodiment, the package is a foil or blister package, labeled ampoule, vial or bottle.

The packaging means of a kit may itself be geared for administration, such as an injection device, an inhaler, syringe, pipette, eye dropper, catheter, cytoscope, trocar, cannula, pressure ejection device, or other such apparatus, from which the formulation may be applied to an affected area of the body, such as the lungs, injected into a subject, delivered to bladder tissue or even applied to and mixed with the other components of the kit.

One or more components of these kits also may be provided in dried or lyophilized forms. When reagents or components are provided as a dried form, reconstitution generally is by the addition of a suitable solvent. It is envisioned that the solvent also may be provided in another package. The kits may include a means for containing the vials or other suitable packaging means in close confinement for commercial sale such as, e.g., injection or blow-molded plastic containers into which the vials are retained. Irrespective of the number or type of packages and as discussed above, the kits also may include, or be packaged with a separate instrument for assisting with the injection/administration or placement of the composition within the body of an animal. Such an instrument may be an inhaler, syringe, pipette, forceps, measuring spoon, eye dropper, catheter, cytoscope, trocar, cannula, pressure-delivery device or any such medically approved delivery means.

The term “treat”, “treating”, or any variation thereof is meant to include therapy utilized to remedy a health problem or condition in a patient or subject. In one embodiment, the health problem or condition may be eliminated permanently or for a short period of time. In another embodiment, the severity of the health problem or condition, or of one or more symptoms characteristic of the health problem or condition, may be lessened permanently, or for a short period of time. The effectiveness of a treatment of pain can be determined using any standard pain index, such as those described herein, or can be determined based on the patient's subjective pain. A patient is considered “treated” if there is a reported reduction in pain or a reduced reaction to stimuli that should cause pain.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention.

### EXAMPLES

Examples are provided below to facilitate a complete understanding of the invention.

### Example 1

### Direct Injection of the Cationic Lipid R-DOTAP Induces a Potent Anti-Tumour Immune Response

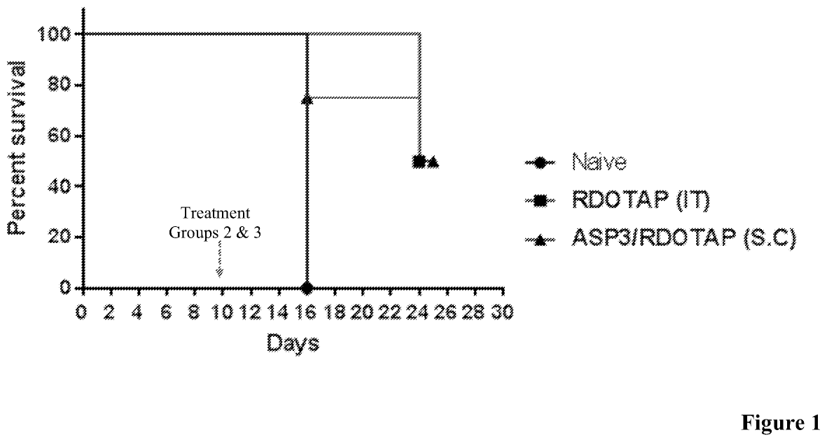

Three groups of mice were injected on day 0 with 50,000 HPV-positive TC-1 tumor cells. In order for a stringent test of antitumor effect, the tumors were allowed to grow to a size of 6-7 mm prior to treatment on Day 10. The aggressively growing tumors reached a size of 10 mm by Day 14. Group 1 mice (Naïve) were left untreated and had to be sacrificed by Day 16. The mice in Group 2 (ASP3/R-DOTAP S.C.) were treated with one subcutaneous injection of R-DOTAP+HPV16(49-57) antigen injection on the opposite flank to the tumor. The mice in Group 3 (R-DOTAP IT) were treated with a single intra-tumoral injection of R-DOTAP only. The tumor sizes and survival of all 3 groups were monitored. In groups 2 and 3 a dramatic slowing of the tumor growth rates were observed (data not shown). The survival plot (FIG. 1) demonstrates the effect of direct injection of R-DOTAP compared to the R-DOTAP.E7 vaccine which has been reported to have potent antitumor efficacy. (See U.S. Pat. Nos. 8,877,206 and 9,789,129 demonstrating the effectiveness of aforementioned therapeutic approach.)

FIG. 1 provides a survival plot: B6 mice (n=4 per group) were implanted with 50,000 TC1 tumor cells subcutaneously. On day 10, group 2 received tumor vaccine R-DOTAP−HPV mix formulation (100 μl) (ASP3-250-HPV mix) containing HPV antigens (ASP3/R-DOTAP (S.C.) in the opposite flank of the tumor and group 3 mice received intra-tumoral injection of R-DOTAP (50 μl of 6 mg/ml) (R-DOTAP (IT)). The results suggest that the cationic lipid without antigen when injected directly is able to promote presentation of antigens expressed within the tumor, and immune activation leading to comparable antitumor efficacy between direct intra-tumoral injection of the cationic lipid only (no antigen) when compared to the proven sub-cutaneous injection of R-DOTAP+antigen.

### Example 2

### Direct Injection of Cationic Lipids into Tumors to Induce Tumor-Specific T Cell and B Cell Responses

To demonstrate intra-tumoral cationic lipid (R-DOTAP, DOTMA) injection will generate antitumor immune responses, mice are to be implanted with syngeneic tumors (TC-1 cells, CT26, A20, etc.) subcutaneously. When the tumors reach 2-4 mm in diameter, tumors will be injected with varying doses of cationic lipids either into the tumor-core or in the tumor periphery using a 30-gauge needle. In a subset of mice, multiple doses (2-3 doses) of cationic lipid at various intervals will be administered. Tumor implanted mice will be euthanized at different times after vaccination to harvest spleen cells and draining lymph nodes. Cell suspensions of lymph node cells, and spleen cells will be co-cultured with known tumor antigen or irradiated tumor cells for 24 hr in an IFN-γ ELISPOT plate. Following this step, Elispot plates will be processed to quantify tumor specific T cell responses. To further assess the polyfunctionality of the T cells, spleen cells are to be co-cultured with antigen or irradiated tumor cells for 12 hr in cell culture media containing protein transport inhibitor. Following this step, the cells will be processed to detect intracellular cytokines (IFN-γ, IL-2, and TNF-α) produced by spleen cells in the co-culture. To evaluate B cell responses induced by R-DOTAP injection, serum will be collected 20-30 days after the first intratumoral injection of R-DOTAP. Serum will be tested for tumor binding antibodies using flow cytometry. The results yielded in conducting these studies, are expected to demonstrate that intra-tumoral cationic lipid administration will induce T cell and B cell responses specific to the tumor.

### Example 3

### Direct Injection of Cationic Lipids into Tumors to Alter the Tumor Microenvironment, Promoting Antitumor Immune Responses

To demonstrate that intratumoral cationic lipid (R-DOTAP, DOTAP racemic mixture, DOTMA, DOEPC, R-DOTAP+HPV16, R-DOTAP+DOPC) injection will have immune-modulatory effects promoting anti-tumor immune responses, mice will be implanted with syngeneic tumors (TC-1 cells, CT26, A20, etc.) subcutaneously. When the tumors reach 2-4 mm in diameter, tumors will be injected with varying doses of cationic lipids into the tumor-core or in the tumor periphery using a 30-gauge needle. The tumors will be isolated from euthanized mice at various times after the first cationic lipid injection and processed to isolate tumor-infiltrating cells. For certain cationic lipids, the phenotypes and gene expression patterns in the tumor-infiltrating cells will be analyzed using multi-Omics technologies such as high-parameter flowcytometry and whole transcriptome analysis at single-cell level. In these studies, we expect to present evidence demonstrating that intratumoral cationic lipid administration will switch tumor microenvironment from a tumor-promoting to a tumor-regressing environment.

### Example 4

### Direct Injection of Cationic Lipids into Tumors to Alter Tumor Growth Characteristics of Distantly Located Tumors

To demonstrate that intra-tumoral injection of cationic lipids, including but not limited to R-DOTAP and DOTMA, will generate systemic antitumor immune responses, mice are to be implanted with syngeneic tumors (TC-1 cells, CT26, A20, etc.) subcutaneously. When the tumors reach 2-4 mm, tumors will be injected with cationic lipids using a 30-gauge needle. At various times after the cationic lipid injections, the tumor-bearing mice will be implanted with a second tumor subcutaneously in a site that is distant from the initial tumor (ex; on the opposite flank), and the growth kinetics of the second implanted tumor will be measured to evaluate systemic antitumor immune responses induced by cationic lipids. In these studies, we expect to present evidence demonstrating that intra-tumoral cationic lipid administration generates antitumor immune responses systemically and is capable of regressing tumors located at distal sites.

### Example 5

### Intra-Tumoral Administration of Cationic Lipids can Synergize with Other Immunotherapy Approaches

To demonstrate the synergy between intra-tumoral cationic lipid injection and other established immunotherapy approaches, studies will be conducted as proposed in Example 1 in a setting where intra-tumoral cationic lipid injection will be used as a combination therapy with other immunotherapy approaches such as checkpoint inhibitor administration and TLR-agonists injection, antitumor cytokines, and/or chemotherapy. In these studies, the results are expected to yield evidence demonstrating that intra-tumoral immunotherapy using cationic lipid induces anti-tumor immune responses that are synergistic with other immunotherapy approaches to promote enhanced tumor regression.

### REFERENCES

¹ Marabelle A., et al. Starting the fight in the tumor: expert recommendations for the development of human intratumoral immunotherapy (HIT-IT). Ann Oncol. 2018; 29(11): 2163-2174
² Arits A H M M, Mosterd K, Essers B A et al. Photodynamic therapy versus topical imiquimod versus topical fluorouracil for treatment of superficial basal-cell carcinoma: a single blind, non-inferiority, randomised controlled trial. Lancet Oncol 2013; 14(7): 647-654.19. Schon M P, Schon M. Imiquimod: mode of action. Br J Dermatol 2007; 157: 8-13
³ Kidner T B, Morton D L, Lee D J et al. Combined intralesional Bacille Calmette-Guerin (BCG) and topical imiquimod for in-transit melanoma. J Immunother 2012; 35(9): 716-720]
⁴ Rook A H, Gelfand J M, Wysocka M et al. Topical resiquimod can induce disease regression and enhance T-cell effector functions in cutaneous Tcell lymphoma. Blood 2015; 126(12): 1452-1461
⁵ Brody J D, Ai W Z, Czerwinski D K et al. In situ vaccination with a TLR9 agonist induces systemic lymphoma regression: a phase I/II study. J Clin Oncol 2010; 28(28): 4324-4332
⁶ Kim Y H, Gratzinger D, Harrison C et al. In situ vaccination against mycosis fungoides by intratumoral injection of a TLR9 agonist combined with radiation: a phase 1-2 study. Blood 2012; 119(2): 355-363
⁷ Marabelle A, Filatenkov A, Sagiv-Barfi I, Kohrt H. Radiotherapy and toll like receptor agonists. Semin Radiat Oncol 2015; 25(1): 34-39
⁸ Formenti S C, Demaria S. Systemic effects of local radiotherapy. Lancet Oncol 2009; 10(7): 718-726; Prise K M, O'Sullivan J M. Radiation-induced bystander signalling in cancer therapy. Nat Rev Cancer 2009; 9(5): 351-360
⁹ Perez C. a, Fu A, Onishko H et al. Radiation induces an antitumour immune response to mouse melanoma. Int J Radiat Biol 2009; 85(12): 1126-1136; Burnette B C, Liang H, Lee Y et al. The efficacy of radiotherapy relies upon induction of type i interferon-dependent innate and adaptive immunity. Cancer Res 2011; 71(7): 2488-2496; Reits E A, Hodge J W, Herberts C A et al. Radiation modulates the peptide repertoire, enhances MEW class I expression, and induces successful antitumor immunotherapy. J Exp Med 2006; 203(5): 1259-1271
¹⁰ Schaue D, Xie M W, Ratikan J A, McBride W H. Regulatory T cells in radiotherapeutic responses. Front Oncol 2012; 2: 90
¹¹ Weide B, Derhovanessian E, Pflugfelder A et al. High response rate after intratumoral treatment with interleukin-2: results from a phase 2 study in 51 patients with metastasized melanoma. Cancer 2010; 116(17): 4139-4146
¹² Weide B, Eigentler T K, Elia G et al. Limited efficacy of intratumoral IL-2 applied to large melanoma metastases. Cancer Immunol Immunother 2014; 63(11): 1231-1232
¹³ Ray A, Williams M A, Meek S M et al. A phase I study of intratumoral ipilimumab and interleukin-2 in patients with advanced melanoma; Oncotarget 2016; 7(39): 64390-64399
¹⁴ Campbell P J, Yachida S, Mudie L J et al. The patterns and dynamics of genomic instability in metastatic pancreatic cancer. Nature 2010; 467(7319): 1109-1113; Gerlinger M, Horswell S, Larkin J et al. Genomic architecture and evolution of clear cell renal cell carcinomas defined by multiregion sequencing. Nat Genet 2014; 46(3): 225-233; Gerlinger M, Rowan A J, Horswell S et al. Intratumor heterogeneity and branched evolution revealed by multiregion sequencing. N Engl J Med 2012; 366(10): 883-892

What is claimed:

1. A method for inducing an anti-tumor immune response by direct intra-tumoral injection of a composition comprising one or more cationic lipids.

2. The method of claim 1, wherein the one or more cationic lipids comprises at least one non-steroidal lipid.

3. The method of claim 1, wherein the one or more cationic lipids comprises 1,2-dioleoyl-3-trim ethyl ammonium propane (DOTAP), N-1-(2,3-dioleoyloxy)-propyl-N,N,N-trimethyl ammonium chloride (DOTMA), 1,2-dioleoyl-sn-glycero-3-ethylphosphocholine (DOEPC), and combinations thereof.

4. The method of claim 3, wherein the cationic lipid comprises an enantiomer of the cationic lipid selected from the group consisting of, R-DOTAP, R-DDA, R-DOEPC, R-DOTMA, S-DOTAP, S-DDA, S-DOEPC, S-DOTMA, variations or analogs thereof.

5. The method of claim 4, wherein the enantiomer is (R)-1,2-dioleoyl-3-trimethylammonium propane (R-DOTAP).

6. The method of claim 1, wherein the composition further comprises one or more antigen.

7. The method of claim 6, wherein the one or more antigen comprises a protein, peptide, polysaccharide, glycoprotein, glycolipid, nucleic acid, or combination thereof.

8. The method of claim 6, wherein the antigen comprises a viral antigen, a bacterial antigen, a pathogenic antigen, microbial antigen, cancer antigen and active fragments, isolates and combinations thereof.

9. The method of claim 6, wherein the antigen comprises a lipoprotein, a lipopeptide, or a protein or peptide modified with an amino acid sequence having an increased hydrophobicity or a decreased hydrophobicity.

10. The method of claim 1, wherein the composition further comprises therapeutic agents and/or pharmaceutically acceptable excipients.

11. The method of claim 1, wherein the composition is in the form of a controlled release preparation.

12. The method of claim 1, wherein the controlled release preparation comprises the use of polymer complexes such as polyesters, polyamino acids, methylcellulose, polyvinyl, poly(lactic acid), and hydrogels.

13. The method of claim 1, wherein antigen-specific CD8+ T cell responses are elevated.

14. A method for inducing an immunogenic response in a subject comprising the intra-tumoral administration of a composition comprising a cationic lipid wherein the administration of the cationic lipid results in the stimulation of an anti-tumor response.

15. The method of claim 14, wherein the cationic lipid comprises 1,2-dioleoyl-3-trim ethyl ammonium propane (DOTAP), N-1-(2,3-dioleoyloxy)-propyl-N,N,N-trimethyl ammonium chloride (DOTMA), 1,2-dioleoyl-sn-glycero-3-ethylphosphocholine (DOEPC), and combinations thereof.

16. The method of claim 15, wherein the cationic lipid comprises (R)-1,2-dioleoyl-3-trimethylammonium propane (R-DOTAP).

17. The method of claim 14, wherein the composition further comprises one or more antigen.

18. The method of claim 17, wherein the one or more antigen comprises a protein, peptide, polysaccharide, glycoprotein, glycolipid, nucleic acid, or combination thereof.

19. The method of claim 14, wherein the composition further comprises therapeutic agents and/or pharmaceutically acceptable excipients.

20. The method of claim 14, wherein the composition is in the form of a controlled release preparation and wherein the controlled release preparation comprises the use of polymer complexes such as polyesters, polyamino acids, methylcellulose, polyvinyl, poly(lactic acid), and hydrogels.
